(19) United States
(12) Patent Application Publication
(10) Pub. No.: US 2019/0302444 A1
(43) Pub. Date: Oct. 3, 2019

(54) ENDOSCOPIC DEVICE, METHOD OF DETERMINING SWITCHING OF OPTICAL SYSTEM IN THE SAME, AND RECORDING MEDIUM

(71) Applicant: OLYMPUS CORPORATION, Tokyo (JP)

(72) Inventor: Tomohiro YOKOHAMA, Tokyo (JP)

(73) Assignee: OLYMPUS CORPORATION, Tokyo (JP)

(21) Appl. No.: 16/365,784

(22) Filed: Mar. 27, 2019

(30) Foreign Application Priority Data
Mar. 28, 2018 (JP) 2018-062018

(51) Int. Cl. G02B 23/24; A61B 1/045; H04N 5/232; H04N 13/218

(52) U.S. Cl. CPC G02B 23/2484; H04N 13/218; H04N 5/23232; A61B 1/045

(57) ABSTRACT

An endoscopic device having an optical path switching part can determine whether or not an optical path is correctly switched by image processing without physically mounting an additional optical path detecting part. The endoscopic device includes a switching part that performs switching between a first optical system and a second optical system, an image pickup device, and a controller. The controller instructs the switching part to switch an optical system, compares, after the switching instruction is given, a plurality of images generated by the image pickup device before and after the switching instruction is given, and determines on the basis of a result of the comparison whether or not the switching is performed normally by the switching part.

### BACKGROUND

### Technical Field

The present invention relates to an endoscopic device, a method of determining switching of optical systems in the endoscopic device, and a recording medium. More particularly, the present invention relates to an endoscopic device having an optical path switching part, a method of determining switching of optical systems in the endoscopic device, and a recording medium.

Priority is claimed on Japanese Patent Application No. 2018-062018, filed Mar. 28, 2018, the content of which is incorporated herein by reference.

### Background Art

Endoscopic devices in which an elongate insertion part is inserted into a specimen and an image of a subject inside the specimen is photographed by an image pickup device provided in a distal part located at a distal end of the insertion part have been widely used in the medical or industrial fields for a long time. For example, in the medical field, a medical endoscopic device is used to insert the insertion part into a body cavity to observe an internal organ in the body cavity or to perform various therapeutic treatments using a treatment tool inserted into a treatment tool channel as needed. For example, in industrial fields, an industrial endoscopic device is used to perform observation or examination of damage or corrosion inside boilers, turbines, engines, chemical plants, and so on.

For example, blades of a high-pressure turbine in a gas turbine engine in a power plant are components in which cracks or the like are easily caused by a thermal shock due to blowing of high-pressure, high-temperature combustion air. Damage such as cracks occurring in the blades is fatal damage in the case of an engine. For this reason, examination or inspection of the blades of a high-pressure turbine using an industrial endoscopic device has become one of the most important items when performing maintenance of a gas turbine engine. In maintenance of a gas turbine engine, a shape of damage is measured in examination of a blade, and it is determined whether or not replacement of the blade is performed on the basis of a result of the measurement.

In examination using an industrial endoscopic device as described above, a stereo measurement technique is widely used as a technique for performing measurement. The stereo measurement technique is a measurement technique for performing measurement on the basis of a 3-dimensional image generated using a parallax between an image equivalent to a right eye and an image equivalent to a left eye by photographing a subject. For this reason, the industrial endoscopic device for performing the stereo measurement includes two objective lenses inside a distal part and has a constitution in which an image corresponding to a subject image formed by an optical system made up of the objective lenses is formed by an image pickup device.

In this case, when the image corresponding to the subject image formed by the objective lens equivalent to the right eye and the image corresponding to the subject image formed by the objective lens equivalent to the left eye are configured to be formed by image pickup devices corresponding to the respective objective lenses, i.e., two image pickup devices, the distal part of the industrial endoscopic device cannot be made thin. Therefore, a constitution in which an image corresponding to a subject image formed by an objective lens equivalent to a right eye and an image corresponding to a subject image formed by an objective lens equivalent to a left eye are formed by a single image pickup device can be considered. However, in the case of this constitution, since an entire imaging area of the single image pickup device is divided to form the image equivalent to the right eye and the image equivalent to the left eye, it can be considered that the resolution of each image is reduced, and measurement accuracy of the stereo measurement in the industrial endoscopic device is reduced.

For this reason, for example, a technique of an endoscopic device in which, while an image equivalent to a right eye and an image equivalent to a left eye are configured to be formed by a single image pickup device, a resolution of each image is raised to improve the measurement accuracy of stereo measurement is proposed as in Japanese Unexamined Patent Application, First Publication No. 2010-128354 (hereinafter referred to as Patent Document 1). The technique disclosed in Patent Document 1 includes a time-division optical path switching part for shielding light from one of optical paths of two optical systems in a time-division manner such that only light from the other optical path of two optical systems is incident upon the single image pickup device. Due to this constitution, in the technique disclosed in Patent Document 1, an image of the light from the optical path on the side that is not shielded is formed on the entire imaging area of the single image pickup device, and a resolution of the image formed by the image pickup device can be raised.

In the technique disclosed in Patent Document 1, the beams of light from the optical paths of the two optical systems are alternately incident upon the image pickup device, and thus the images corresponding to the subject images formed by the respective optical systems can be formed to perform the stereo measurement. Thus, in the industrial endoscopic device to which the technique disclosed in Patent Document 1 is applied, the resolution of each of the photographed images equivalent to the right eye and the photographed images equivalent to the left eye is raised for the purpose of performing the stereo measurement, so that the measurement accuracy when the subject within a specimen is measured can be improved.

In the industrial endoscopic device to which the technique disclosed in Patent Document 1 is applied, as the time-division optical path switching part for switching the optical paths of the two optical systems, providing a component for alternately shielding the optical paths in a time-division manner at a distal part of the industrial endoscopic device is essential.

A constitution in which, as the time-division optical path switching part, a shield member for shielding the light from any one of the optical paths is rotated about an axis is disclosed in Patent Document 1. That is, a mechanical mechanism that physically moves the shield member is disclosed in Patent Document 1.

For the optical path switching part, it is presupposed that one of the plurality of optical paths can be reliably switched. In the mechanical mechanism disclosed in Patent Document 1, the optical path is switched by physically moving the shield member. For example, in the case of a mechanism in which a shading member is moved by a magnetic drive device, the mechanism has a physical actuator in a distal part thereof.

### SUMMARY

An endoscopic device according to a first aspect of the present invention includes: a switching part configured to switch optical systems such that only any one of a first subject image of a subject which is formed by light passing through a first optical system and a second subject image of the subject which is formed by light passing through a second optical system is formed in an image formation area in which the first subject image and the second subject image are formed in common; an image pickup device configured to photograph the first and second subject images formed in the image formation area to generate an image; a driver configured to output a switching signal to the switching part, the switching signal being configured to switch the first optical system to the second optical system or switch the second optical system to the first optical system; and a controller. The controller gives the driver an instruction to output the switching signal, compares, after the instruction is given, a plurality of images generated by the image pickup device before and after the instruction is given, and determines on the basis of a result of the comparison whether or not the switching is performed normally by the switching part.

The controller may be configured to calculate, after the instruction is given, an amount of difference between a first image generated by the image pickup device before the instruction is given and a second image generated by the image pickup device after the instruction is given, compare the amount of difference and a prescribed threshold value, and as a result of the comparison, in a case where the amount of difference is greater than the prescribed threshold value, determine that the switching is performed normally by the switching part.

After the instruction is given, the controller may compare a first image generated by the image pickup device before the instruction is given, a second image generated by the image pickup device after the instruction is given, and a third image generated by the image pickup device after the second image is generated.

The controller may be configured to compare an amount of difference between the first image and the second image, an amount of difference between the first image and the third image, and an amount of difference between the second image and the third image, and as a result of the comparison, in a case where the amount of difference between the second image and the third image is a minimum, determine that the switching is performed normally by the switching part.

After the instruction is given, the controller may compare a first image generated by the image pickup device before the instruction is given, a second image generated by the image pickup device after the instruction is given, and a third image generated by the image pickup device before the first image is generated.

The controller may be configured to compare an amount of difference between the first image and the second image, an amount of difference between the first image and the third image, and an amount of difference between the second image and the third image, and as a result of the comparison, in a case where the amount of difference between the first image and the second image is a minimum, determine that the switching is performed normally by the switching part.

After the instruction is given, the controller may compare an average brightness value of the images generated by the image pickup device before and after the instruction is given with a prescribed threshold value, and as a result of the comparison, in a case where the average brightness value of all the images is smaller than the prescribed threshold value, determine that the switching is not performed normally by the switching part.

After the instruction is given, the controller may compare contrasts of the images generated by the image pickup device before and after the instruction is given.

After the instruction is given, the controller may compare average exposure intensities of the images generated by the image pickup device before and after the instruction is given.

After the instruction is given, the controller may compare average hues of the images generated by the image pickup device before and after the instruction is given.

After the instruction is given, the controller may compare left or right average brightnesses of the images generated by the image pickup device before and after the instruction is given.

After the instruction is given, the controller may compare left or right average colors of the images generated by the image pickup device before and after the instruction is given.

The controller may be configured to compare, after the instruction is given, two or more of average exposure intensities, contrasts, average hues, left or right average brightness, and left or right average colors of the images generated by the image pickup device before and after the instruction is given, and determine, on the basis of a result of the comparison, whether or not the switching is performed normally by the switching part.

A distal part of the endoscopic device having the first and the second optical systems may include a type determiner that includes information for identifying a type; and the endoscopic device may further include a type detector that reads information about the type determiner and determines a type of the distal part.

In a case where the type detector determines that the first subject image and the second subject image are different in focal position from each other in the distal part, after the instruction is given, the controller may compare contrasts of the images generated by the image pickup device before and after the instruction is given.

In a case where the type detector determines that the first subject image and the second subject image are different in aperture value from each other in the distal part, after the instruction is given, the controller may compare average exposure intensities of the images generated by the image pickup device before and after the instruction is given.

In a case where the type detector determines that the first subject image and the second subject image have respective parallaxes in the distal part, light from the first optical system may be obliquely incident upon the image formation area from a right side, and light from the second optical system may be obliquely incident upon the image formation area from a left side, after the instruction is given, the controller may compare left or right average brightnesses of the images generated by the image pickup device before and after the instruction is given.

In a case where the type detector determines that the first optical system and the second optical system are different in spectral transmissivity from each other in the distal part, after the instruction is given, the controller may compare average colors of the images generated by the image pickup device before and after the instruction is given.

In a case where the type detector determines that the first optical system and the second optical system are different in polarization transmissivity from each other in the distal part, after the instruction is given, the controller may compare average brightnesses of the images generated by the image pickup device before and after the instruction is given.

In a case where the type detector determines that the first subject image and the second subject image have respective parallaxes in the distal part, light from the first optical system may be obliquely incident upon the image formation area from a right side, and light from the second optical system may be obliquely incident upon the image formation area from a left side, after the instruction is given, the controller may compare left or right average colors of the images generated by the image pickup device before and after the instruction is given.

The controller may further include a storage that stores the images generated by the image pickup device before and after the instruction is given.

A method of determining switching of optical systems in the endoscopic device according to the first aspect of the present invention is a method of determining switching of optical systems in the endoscopic device that includes a switching part configured to switch the optical systems such that only any one of a first subject image of a subject which is formed by light passing through a first optical system and a second subject image of the subject which is formed by light passing through a second optical system is formed in an image formation area in which the first subject image and the second subject image are formed in common, and an image pickup device configured to photograph the first and second subject images formed in the image formation area to generate an image, and includes: a switching step of instructing the switching part to switch the first optical system to the second optical system or to switch the second optical system to the first optical system; a step of, after the switching step, comparing a plurality of images generated by the image pickup device before and after the switching step; and a step of determining on the basis of a result of the comparison whether or not the switching is performed normally by the switching part.

A program according to a first aspect of the present invention is a program for controlling an endoscopic device that includes a switching part configured to switch optical systems such that only any one of a first subject image of a subject which is formed by light passing through a first optical system and a second subject image of the subject which is formed by light passing through a second optical system is formed in an image formation area in which the first subject image and the second subject image are formed in common, an image pickup device configured to photograph the first and second subject images formed in the image formation area to generate an image, and a controller. The program causes the controller to execute: a switching step of instructing the switching part to switch the first optical system to the second optical system or to switch the second optical system to the first optical system; a step of, after the switching step, comparing a plurality of images generated by the image pickup device before and after the switching step; and a step of determining on the basis of a result of the comparison whether or not the switching is performed normally by the switching part.

A recording medium according to a first aspect of the present invention is a computer-readable recording medium that records a program for controlling an endoscopic device that includes a switching part configured to switch optical systems such that only any one of a first subject image of a subject which is formed by light passing through a first optical system and a second subject image of the subject which is formed by light passing through a second optical system is formed in an image formation area in which the first subject image and the second subject image are formed in common, an image pickup device configured to photograph the first and second subject images formed in the image formation area to generate an image, and a controller. The program causes the controller of the endoscopic device to execute: a switching step of instructing the switching part to switch the first optical system to the second optical system or to switch the second optical system to the first optical system; a step of, after the switching step, comparing a plurality of images generated by the image pickup device before and after the switching step; and a step of determining on the basis of a result of the comparison whether or not the switching is performed normally by the switching part.

### BRIEF DESCRIPTION OF THE DRAWINGS

FIG. 1 is a block diagram illustrating a whole constitution of an endoscopic device according to a first embodiment of the present invention.

FIG. 2 is a timing chart illustrating switching of a photographed image when an optical path is switched in the endoscopic device according to the first embodiment of the present invention.

FIG. 3 is a flow chart illustrating an operation of the endoscopic device according to the first embodiment when the optical path is switched.

FIG. 4 is a block diagram illustrating a whole constitution of an endoscopic device according to a second embodiment of the present invention.

FIG. 5 is a timing chart illustrating switching of a photographed image when an optical path is switched in the endoscopic device according to the second embodiment of the present invention.

FIG. 6 is a flow chart illustrating an operation of the endoscopic device according to the second embodiment when the optical path is switched.

FIG. 7 is a block diagram illustrating a whole constitution of an endoscopic device according to a third embodiment of the present invention.

FIG. 8 is a flow chart illustrating an operation of the endoscopic device according to the third embodiment when the optical path is switched.

FIG. 9 is a block diagram illustrating a whole constitution of an endoscopic device according to a fourth embodiment of the present invention.

FIG. 10 is a graph illustrating a brightness profile of an image of the endoscopic device according to the fourth embodiment of the present invention.

FIG. 11 is a flow chart illustrating an operation of the endoscopic device according to the fourth embodiment when the optical path is switched.

### DETAILED DESCRIPTION OF THE PREFERRED EMBODIMENTS

Hereinafter, an endoscopic device according to an embodiment of the present invention will be described in detail with reference to the drawings.

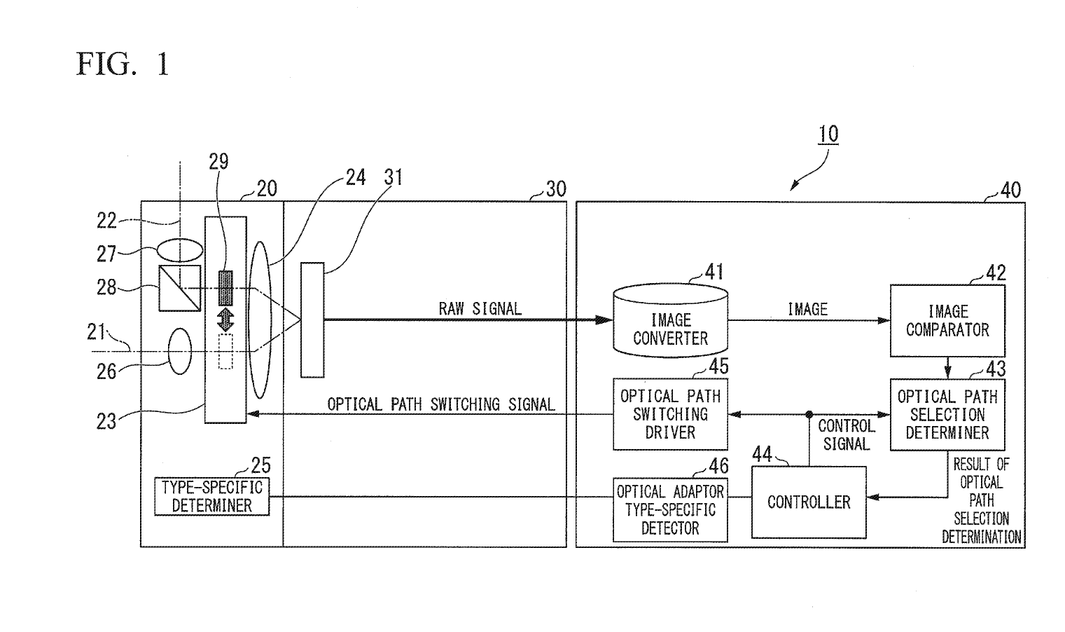

First, an endoscopic device according to a first embodiment of the present invention will be described. FIG. 1 is a block diagram illustrating a whole constitution of the endoscopic device according to the first embodiment of the present invention. The endoscopic device 10 includes a distal part (an optical adaptor) 20 that is inserted into a specimen, an imaging part 30 that forms an image, and a main body part 40 that controls the entire endoscopic device. The distal part 20 is connected to a distal end of the imaging part 30, and may be attachable/detachable. The imaging part 30 and the main body part 40 are connected by an elongate insertion part (not shown) or the like that is inserted into the specimen. The imaging part 30 includes an imager (an image pickup device) 31.

The distal part 20 includes a first objective optical system 21, a second objective optical system 22, an optical path switching part 23, and a common optical system 24. The distal part 20 may or may not include an optical adaptor type determiner 25. In the example of FIG. 1, a direction of an optical axis of the first objective optical system is parallel to that of the common optical system 24, and a direction of an optical axis of the second objective optical system is perpendicular to that of the common optical system 24. For this reason, the first objective optical system 21 includes a lens 26, and the second objective optical system 22 includes a lens 27 and a prism 28.

The optical path switching part 23 performs switching between the first objective optical system (the first optical path) 21 and the second objective optical system (the second optical path) 22. That is, the optical path switching part 23 performs switching between a beam of light entering from the first objective optical system 21 and a beam of light entering from the second objective optical system 22 and causes only one of the two beams of light to be incident upon the imager 31 via the common optical system 24. That is, either a first subject image of a subject which is formed by the light entering from the first objective optical system 21 or a second subject image of the subject which is formed by the light entering from the second objective optical system 22 is formed by the imager 31.

The optical path switching part 23 includes, for example, a shading member 29, and cuts off the light entering from the objective optical system on an unselected side by inserting the shading member 29 into the objective optical system on the unselected side. The shading member 29 is driven, for example, by a magnetic drive device.

Although an example in which field-of-view directions of the first and second objective optical systems 21 and 22 are different from each other is shown in FIG. 1, the present embodiment is not limited thereto. The present embodiment can be applied to all cases where the number of optical paths (objective optical systems) is two or more, and the optical path is switched by inserting/ejecting any member into/from the optical path.

The image formed by the imager 31 is converted into a RAW signal, and is transmitted to the main body part 40. The main body part 40 includes an image converter 41, an image comparator 42, an optical path selection determiner 43, a controller 44, and an optical path switching driver 45. The main body part 40 may include an optical adaptor type detector 46.

The image converter (the storage) 41 converts the RAW signal transmitted from the imager 31, and accumulates a result of the conversion as an image. The image comparator 42 compares a plurality of images accumulated by the image converter 41. To be specific, when an image formed before the optical path is switched is set as a first image and an image formed after the optical path is switched is set as a second image, the image comparator 42 compares the first image and the second image.

The optical path selection determiner 43 determines, on the basis of a result of the comparison between the images using the image comparator 42, to which optical path the optical path is switched now or whether or not the optical path is correctly switched (optical path selection determination) and transmits a result of the optical path selection determination to the controller 44. The controller 44 transmits a control signal for controlling the optical path selection determiner 43 and the optical path switching driver 45 on the basis of the result of the optical path selection determination at the optical path selection determiner 43 and controls the switching of the optical path. The optical path switching driver 45 transmits an optical path switching signal for instructing the optical path switching part 23 to switch the optical path to the optical path switching part 23 on the basis of the control signal from the controller 44.

The optical adaptor type detector 46 reads information (data) about the optical adaptor type determiner 25 of the distal part (the optical adaptor) 20 and determines the type of the distal part (the optical adaptor) 20 on the basis of the data.

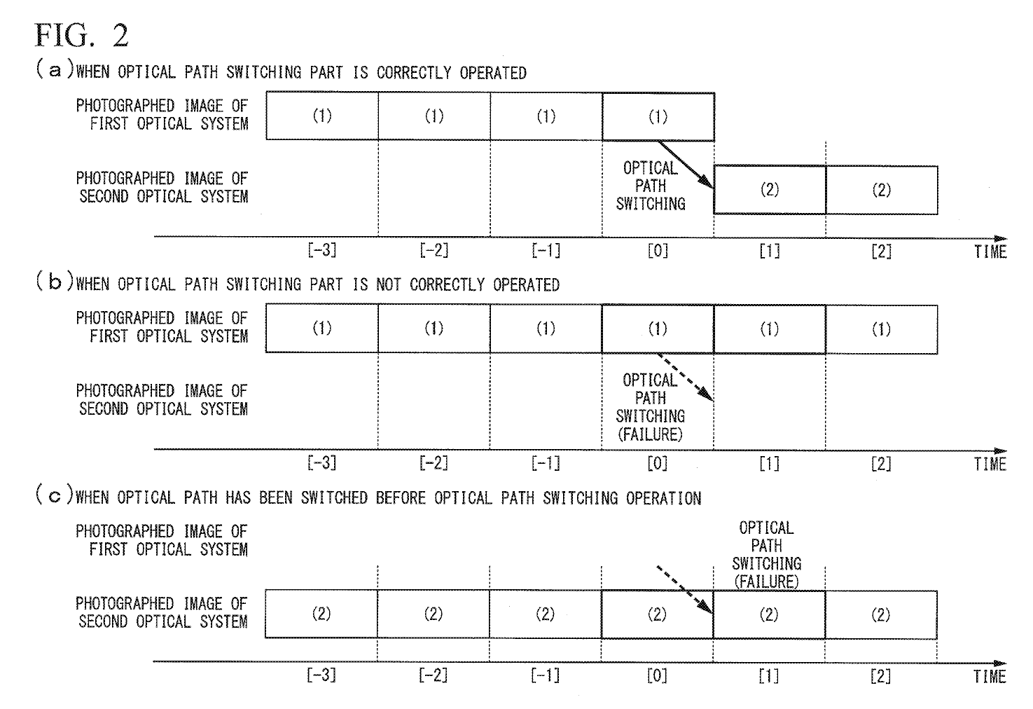

A mechanism of the optical path selection determination in the image comparator 42 will be described. FIG. 2 is a timing chart illustrating switching of a photographed image when an optical path is switched. The horizontal axis of FIG. 2 indicates time, and the number of a frame photographed at each time is given between square brackets ([ ]). The optical path switching part 23 switches an optical path from the first objective optical system 21 to the second objective optical system 22 at a time of the boundary between a 0^{th} frame and a 1^{st} frame. That is, the optical path switching driver 45 transmits an optical path switching signal for instructing the optical path switching part 23 to switch an optical path to the optical path switching part 23 at the time of the boundary between the 0^{th} frame and the 1^{st} frame. The instruction of the optical path switching signal may include an instruction about whether the optical path is switched to the first objective optical system 21 or the second objective optical system 22.

In the timing charts of FIGS. 2(A) to 2(C), an upper stage indicates the first subject image of the subject which is formed and obtained by the light entering from the first objective optical system 21 as a photographed image (1). A lower stage indicates the second subject image of the subject which is formed and obtained by the light entering from the second objective optical system 22 as a photographed image (2).

FIG. 2(A) is a timing chart illustrating a case where the optical path switching part 23 is correctly operated. When the optical path switching part 23 is correctly operated, the optical path is switched, so that, as illustrated in FIG. 2(A), the image of the 0^{th} frame is the first subject image (1) of the subject which is formed and obtained by the light entering from the first objective optical system 21, and the image of the 1^{st} frame is the second subject image (2) of the subject which is formed and obtained by the light entering from the second objective optical system 22.

FIG. 2(B) is a timing chart illustrating a case where the optical path switching part 23 is not correctly operated. In the case where the optical path switching part 23 is not correctly operated, the optical path is not switched, and as illustrated in FIG. 2(B), the image of the 1^{st} frame is also the first subject image (1) of the subject which is formed and obtained by the light entering from the first objective optical system 21.

FIG. 2(C) is a timing chart illustrating a case where an optical path is switched before an optical path switching operation. For example, the optical path may be switched by an external pressure or the like before an optical path switching operation. In this case, as illustrated in FIG. 2(C), an image of a frame before the optical path switching operation is the second subject image (2) of the subject which is formed and obtained by the light entering from the second objective optical system 22. Therefore, the optical path is not switched by the optical path switching operation.

The image converter 41 accumulates the image of the 0^{th} frame (the first image) and the image of the 1^{st} frame (the second image), and outputs them to the image comparator 42. The image comparator 42 compares the image of the 0^{th} frame and the image of the 1^{st} frame using a general image comparing method such as the sum of absolute differences (SAD), the sum of squared differences (SSD), the zero-means normalized cross-correlation (ZNCC), or the like. The image comparator 42 may calculate luminance, brightness, contrast, color difference, and so on from each of the images as evaluation values, and compare the evaluation values instead of directly comparing data of the two images.

In this case, if a frame rate is sufficiently high, an amount of difference between two images obtained by the same optical system is smaller than that between two images obtained by different optical systems. Therefore, in a case where an amount of difference between the image of the 0^{th} frame and the image of the 1^{st} frame is greater than a prescribed threshold value, the optical path selection determiner 43 determines that the compared images are obtained by different optical systems. That is, as illustrated in FIG. 2(A), the optical path selection determiner 43 determines that the optical path is switched at the time of the boundary between the 0^{th} frame and the 1^{st} frame.

In FIG. 2(A), the optical path switching driver 45 outputs the optical path switching signal for switching the optical path to the second objective optical system 22. Therefore, since the optical path selection determiner 43 determines that the images can be formed by the different optical systems before and after the time when the optical path switching signal is output, the optical path selection determiner 43 can determine that the image obtained after the optical path is switched is the second subject image (2) of the subject which is formed and obtained by the light entering from the second objective optical system 22. That is, which optical path is currently used can be determined based on two criteria: an instruction from the optical path switching driver 45 on switching of the optical path, and whether or not the optical path selection determiner 43 determines that the image is switched.

On the other hand, in a case where the amount of difference between the image of the 0^{th} frame and the image of the 1^{st} frame is smaller than the prescribed threshold value, the optical path selection determiner 43 determines that the compared images are obtained by the same optical system. That is, the optical path selection determiner 43 determines that the optical path is not switched at the time of the boundary between the 0^{th} frame and the 1^{st} frame. This case includes a case where the optical path switching part 23 is not correctly operated as illustrated in FIG. 2(B), and a case where the optical path has been switched before the optical path switching operation as illustrated in FIG. 2(C). For this reason, it cannot be determined which optical path is currently used.

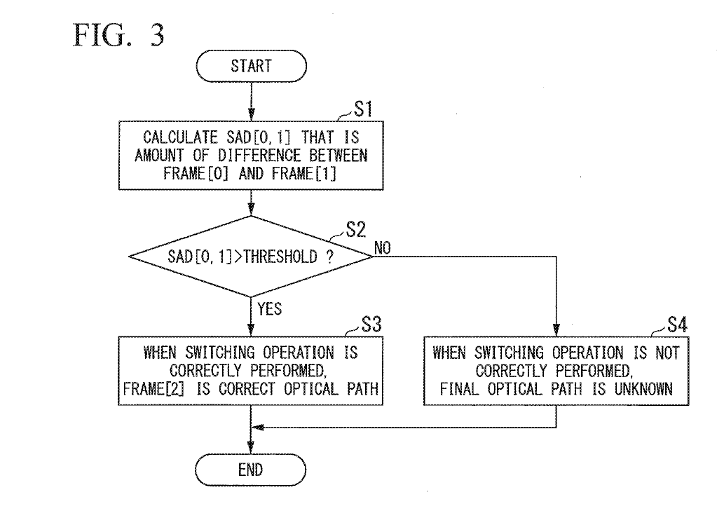

FIG. 3 is a flow chart illustrating an operation of the endoscopic device according to the present embodiment when the optical path is switched. Here, an example in which the sum of absolute differences (SAD) is used as an image comparing method is given. When the optical path switching part 23 switches the optical path at the time of the boundary between the 0^{th} frame and the 1^{st} frame, an operation flow of the image comparator 42 is initiated. First, in step S1, the image comparator 42 calculates SAD[0,1] that is an amount of difference between image data of the 0^{th} frame and image data of the 1^{st} frame according to SAD.

Next, in step S2, the image comparator 42 determines whether or not the calculated SAD[0,1] is greater than a prescribed threshold value. In a case where the SAD[0,1] is greater than the prescribed threshold value, the process proceeds to step S3, and the optical path selection determiner 43 determines that a switching operation is performed normally.

Therefore, an image of a 2^{nd} frame is understood to be an image that passes through the correct optical path. The operation flow when the optical path is switched is thus completed.

In a case where the SAD[0,1] is smaller than or equal to the prescribed threshold value, the process proceeds to step S4, and the optical path selection determiner 43 determines that the switching operation is not performed normally. In this case, the current optical path is unknown. The operation flow when the optical path is switched is thus completed.

As described above, in the present embodiment, in the endoscopic device having the optical path switching part, the two images before and after the optical path is switched (the first and second images) are compared, and it is determined whether or not there is a difference between the two images. It is determined from this result whether or not the optical path is correctly switched. Therefore, even if no additional optical path detecting part is physically mounted, it can be determined whether or not the optical path is correctly switched by image processing.

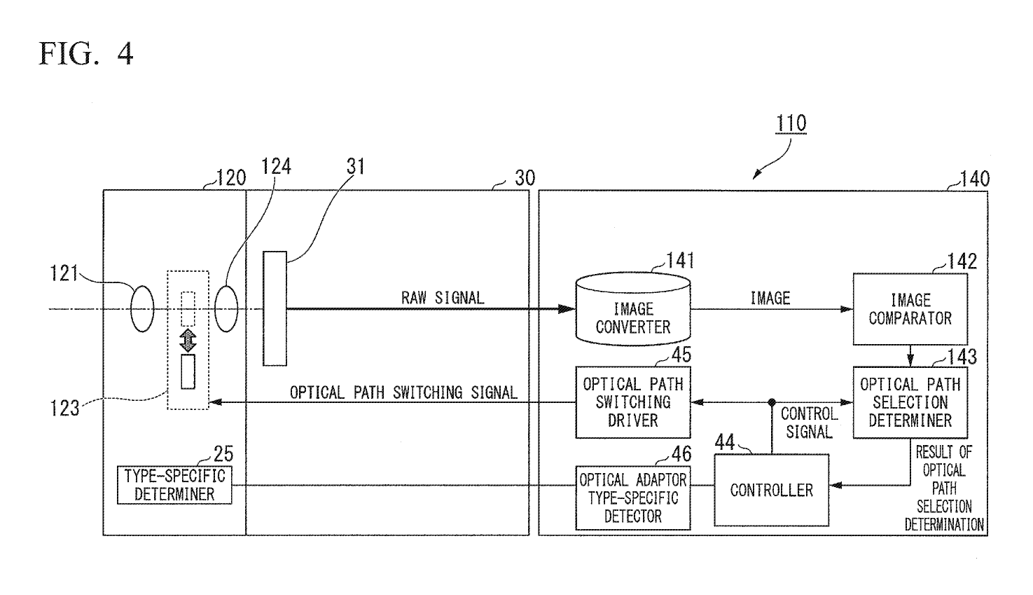

Next, an endoscopic device according to a second embodiment of the present invention will be described. FIG. 4 is a block diagram illustrating a whole constitution of an endoscopic device according to a second embodiment of the present invention. An endoscopic device 110 includes a distal part (an optical adaptor) 120 that is inserted into a specimen, an imaging part 30 that forms an image, and a main body part 140 that controls the entire endoscopic device. The distal part 120 is connected to a distal end of the imaging part 30, and may be attachable/detachable. The imaging part 30 and the main body part 140 are connected by an elongate insertion part (not shown) or the like that is inserted into the specimen. The imaging part 30 includes an imager (an image pickup device) 31.

The distal part 120 includes a common objective optical system 121, an optical path switching part 123, and a common optical system 124. The distal part 120 may or may not include an optical adaptor type determiner 25.

The optical path switching part 123 includes, for example, an optical member 129 and performs switching between a first objective optical system (a first optical path) and a second objective optical system (a second optical path) by inserting/ejecting the optical member 129 into/from the optical path. The optical member 129 is, for example, a filter such as a parallel flat plate, and focal lengths of the first and second objective optical systems are different from each other. That is, since the optical member 129 is inserted into/ejected from the optical path, an angle of view is switched. The optical member 129 is driven, for example, by a magnetic drive device.

In the case where the optical member 129 is inserted into the optical path, the optical path (the first optical path) is set as the first objective optical system. In the case where the optical member 129 is not inserted into the optical path, the optical path (the second optical path) is set as the second objective optical system. The optical path switching part 123 performs switching between a beam of light entering from the first objective optical system and a beam of light entering from the second objective optical system and causes only one of the two beams of light to be incident upon the imager 31 via the common optical system 124. That is, either a first subject image of a subject which is formed by the light entering from the first objective optical system or a second subject image of the subject which is formed by the light entering from the second objective optical system is formed by the imager 31.

In the example of FIG. 4, a direction of an optical axis of the first objective optical system and a direction of an optical axis of the second objective optical system are the same as that of the common optical system 124. A difference between the first objective optical system and the second objective optical system is merely whether or not the optical member 129 is on the optical path.

The image formed by the imager 31 is converted into a RAW signal and is transmitted to the main body part 140. The main body part 140 includes an image converter 141, an image comparator 142, an optical path selection determiner 143, a controller 44, and an optical path switching driver 45. The main body part 140 may include an optical adaptor type detector 46.

The image converter (the storage) 141 converts the RAW signal transmitted from the imager 31, and accumulates a result of the conversion as an image. The image comparator 142 compares a plurality of images accumulated by the image converter 141. To be specific, when an image of a single frame which is formed before the optical path is switched is set as a first image and images of two frames which are formed after the optical path is switched are set as second and third images, the image comparator 142 compares the first image and the second image, the second image and the third image, and the first image and the third image.

The optical path selection determiner 143 determines on the basis of a result of the comparison between the images at the image comparator 142 to which optical path the optical path is currently switched or whether or not the optical path is correctly switched (optical path selection determination) and transmits a result of the optical path selection determination to the controller 44. The controller 44 transmits a control signal for controlling the optical path selection determiner 143 and the optical path switching driver 45 on the basis of the result of the optical path selection determination at the optical path selection determiner 143 and controls the switching of the optical path. The optical path switching driver 45 transmits an optical path switching signal for instructing the optical path switching part 123 to switch the optical path to the optical path switching part 123 on the basis of the control signal from the controller 44.

The optical adaptor type detector 46 reads information (data) about the optical adaptor type determiner 25 of the distal part (the optical adaptor) 120 and determines a type of the distal part (the optical adaptor) 120 on the basis of the data.

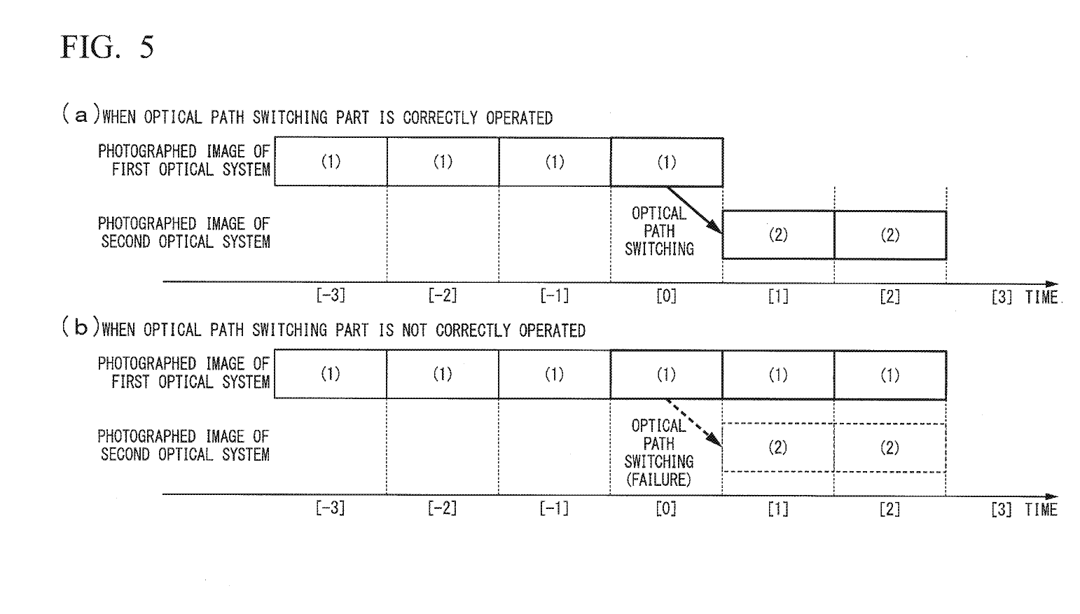

A mechanism of the optical path selection determination at the image comparator 142 will be described. FIG. 5 is a timing chart illustrating switching of a photographed image when an optical path is switched. The horizontal axis of FIG. 5 indicates time, and the number of a frame photographed at each time is given between square brackets (H). The optical path switching part 123 switches an optical path from the first objective optical system to the second objective optical system at a time of the boundary between a 0^{th} frame and a 1^{st} frame. That is, the optical path switching driver 45 transmits an optical path switching signal for instructing the optical path switching part 123 to switch an optical path to the optical path switching part 123 at the time of the boundary between the 0^{th} frame and the 1^{st} frame. The instruction of the optical path switching signal may include an instruction about whether the optical path is switched to the first objective optical system or the second objective optical system.

In the timing charts of FIGS. 5(A) and 5(B), an upper stage indicates the first subject image of the subject which is formed and obtained by the light entering from the first objective optical system as a photographed image (1). A lower stage indicates the second subject image of the subject which is formed and obtained by the light entering from the second objective optical system as a photographed image (2).

FIG. 5(A) is a timing chart illustrating a case where the optical path switching part 123 is correctly operated. When the optical path switching part 123 is correctly operated, the optical path is switched, so that, as illustrated in FIG. 5(A), the image of the 0^{th} frame is the first subject image (1) of the subject which is formed and obtained by the light entering from the first objective optical system, and the images of the 1^{st} and subsequent frames are the second subject images (2) of the subject which are formed and obtained by the light entering from the second objective optical system.

FIG. 5(B) is a timing chart illustrating a case where the optical path switching part 123 is not correctly operated. In the case where the optical path switching part 123 is not correctly operated, the optical path is not switched, and as illustrated in FIG. 5(B), the images of the 1^{st} and subsequent frames also become the first subject images (1) of the subject which are formed and obtained by the light entering from the first objective optical system.

The image converter 141 accumulates the image of the 0^{th} frame (the first image), the image of the 1^{st} frame (the second image), and the image of the 2^{nd} frame (the third image) and outputs them to the image comparator 142. The image comparator 142 compares the image of the 0^{th} frame and the image of the 1^{st} frame, and the image of the 1^{st} frame and the image of the 2^{nd} frame using a general image comparing method such as the sum of absolute differences (SAD), the sum of squared differences (SSD), the zero-means normalized cross-correlation (ZNCC), or the like. The image comparator 142 may calculate luminance, brightness, contrast, color difference, and so on from each of the images as evaluation values, and compare the evaluation values instead of directly comparing data of the two images.

In this case, if a frame rate is sufficiently high, an amount of difference between two images obtained by the same optical system is smaller than that between two images obtained by different optical systems. Thus, in a case where the optical path switching part 123 is correctly operated, an amount of difference between the image of the 1^{st} frame and the image of the 2^{nd} frame is minimized Therefore, in a case where an amount of difference between the image of the 1^{st} frame and the image of the 2^{nd} frame is smaller than that between the image of the 0^{th} frame and the image of the 1^{st} frame, the optical path selection determiner 143 determines that the image of the 0^{th} frame and the image of the 1^{st} frame are obtained by the different optical systems. That is, as illustrated in FIG. 5(A), the optical path selection determiner 143 determines that the optical path is switched at the time of the boundary between the 0^{th} frame and the 1^{st} frame.

In FIG. 5(A), the optical path switching driver 45 outputs the optical path switching signal for switching the optical path to the second objective optical system. Therefore, since the optical path selection determiner 143 determines that the images can be formed by the different optical systems before and after the time when the optical path switching signal is output, the optical path selection determiner 143 can determine that the image obtained after the optical path is switched is the second subject image (2) of the subject which is formed and obtained by the light entering from the second objective optical system. That is, which optical path is used now can be determined based on two criteria: an instruction from the optical path switching driver 45 on switching of the optical path and whether or not the optical path selection determiner 143 determines that the image is switched.

On the other hand, in a case where the optical path switching part 123 is not correctly operated or the optical path has already been switched before being switched, the three images are also obtained by the same optical system. Thus, even if any two of the three images are taken, an amount of difference between any two images is the same as that between other two images. Accordingly, if the amount of difference between the image of the 1^{st} frame and the image of the 2^{nd} frame among those between the two images selected from the three images is a minimum value, the optical path selection determiner 143 determines that the optical system is correctly switched. That is, the optical path selection determiner 143 determines that the optical path is not switched at the time of the boundary between the 0^{th} frame and the 1^{st} frame. This case includes a case where the optical path switching part 123 is not correctly operated (FIG. 5(B)), and a case where the optical path has been switched before the optical path switching operation (not shown). For this reason, it cannot be determined which optical path is used now.

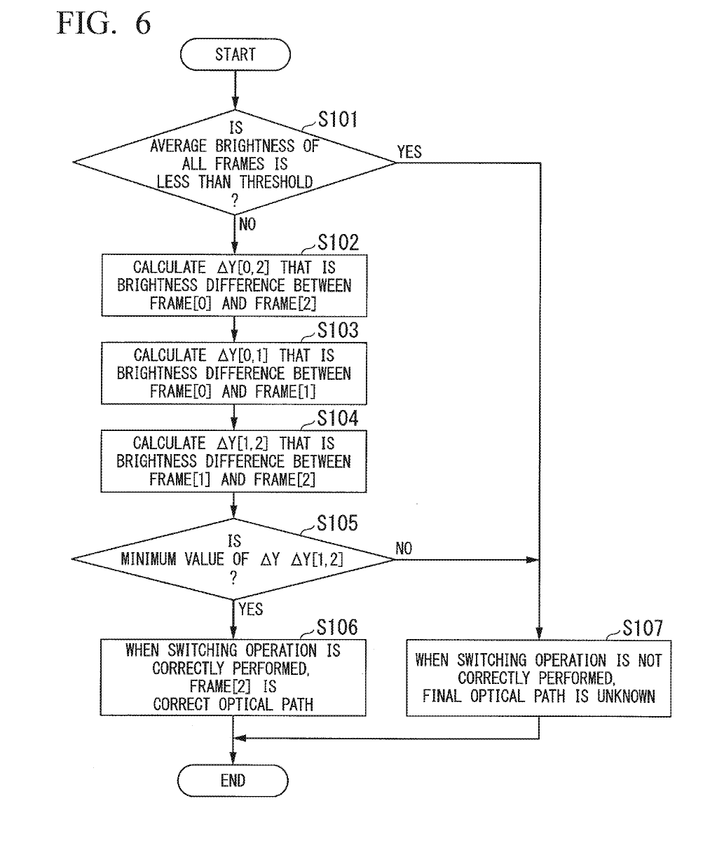

FIG. 6 is a flow chart illustrating an operation of the endoscopic device according to the present embodiment when the optical path is switched. Here, an example in which sum of absolute differences (SAD) is used as an image comparing method is given. When the optical path switching part 123 switches the optical path at the time of the boundary between the 0^{th} frame and the 1^{st} frame, an operation flow of the image comparator 42 is initiated.

First, in step S101, the image comparator 142 determines whether or not average brightness of the obtained images of all the frames (here, the 0^{th} frame, the 1^{st} frame, and the 2^{nd} frame) is smaller than a prescribed threshold value. A case where the images are completely dark is excluded by step S101. Here, the average brightness is not a brightness average of all pixels within an image, and may designate a partial area within an image to take a brightness average of pixels within the area.

In a case where the average brightness of the images of all the frames is greater than or equal to the prescribed threshold value, the process proceeds to step S102. In the case where the average brightness of the images of all the frames is smaller than the prescribed threshold value (the images are completely dark), the process proceeds to step S107.

In step S102, the image comparator 142 calculates SAD[0,2] that is an amount of difference between image data of the 0^{th} frame and image data of the 2^{nd} frame according to SAD. This process proceeds to step S103, and the image comparator 142 calculates SAD[0,1] that is an amount of difference between image data of the 0^{th} frame and image data of the 1^{st} frame according to SAD. This process proceeds to step S104, and the image comparator 142 calculates SAD[1,2] that is an amount of difference between the image data of the 1^{st} frame and image data of the 2^{nd} frame according to SAD.

Next, in step S105, the image comparator 142 determines whether or not a minimum among the calculated SAD[0,2], SAD[0,1], and SAD[1,2] is SAD[1,2]. In a case where SAD[1,2] is the minimum, the process proceeds to step S106, and the optical path selection determiner 143 determines that the switching operation is performed normally. Therefore, the image of the 2^{nd} frame is understood be an image that passes through the correct optical path. The operation flow when the optical path is switched is thus completed.

In a case where SAD[1,2] is not the minimum, the process proceeds to step S107. In step S107, the optical path selection determiner 143 determines that the switching operation is not performed normally. In this case, the current optical path is unknown. The operation flow when the optical path is switched is thus completed.

In the aforementioned description, although the image of the 2^{nd} frame is used in addition to the image of the 0^{th} frame and the image of the 1^{st} frame, the present embodiment is not limited thereto. In addition to enabling the calculation of the amounts of difference between the two images before and after the optical path is switched, it is desirable that the amount of difference between the two images before the optical path is switched or the amount of difference between the two images after the optical path is switched can be calculated.

As described above, in the present embodiment, in the endoscopic device having the optical path switching part, the amounts of difference between the two images selected from among the three images (the first, second, and third images) before and after the optical path is switched are calculated and compared, and it is determined from this result whether or not the optical path is correctly switched. Therefore, even if an additional optical path detecting part is physically mounted, it can be determined whether or not the optical path is correctly switched by image processing. Further, in the present embodiment, since the amounts of difference need not be compared with the prescribed threshold value, the prescribed threshold value need not be adjusted to each subject.

In the aforementioned description, although the example in which the focal lengths of the first and second objective optical systems are different from each other (the angles of view are different from each other), and the optical path is switched by the insertion/ejection of the optical member is given, the present embodiment is not limited thereto.

For example, in a case where an optical member such as a parallel flat plate that changes an optical path length is inserted/ejected, the first and second objective optical systems are different in focus position (distance) from each other. In this case, the first and second objective optical systems have the same F-values and focal lengths. In this case, it can be determined whether the optical path is correctly switched using an amount of difference between contrasts (whether or not to be resolved) of the two images. In this way, the present embodiment can be applied to all cases where the number of optical paths (objective optical systems) is two or more, and the optical path is switched by inserting/ejecting any member into/from the optical path.

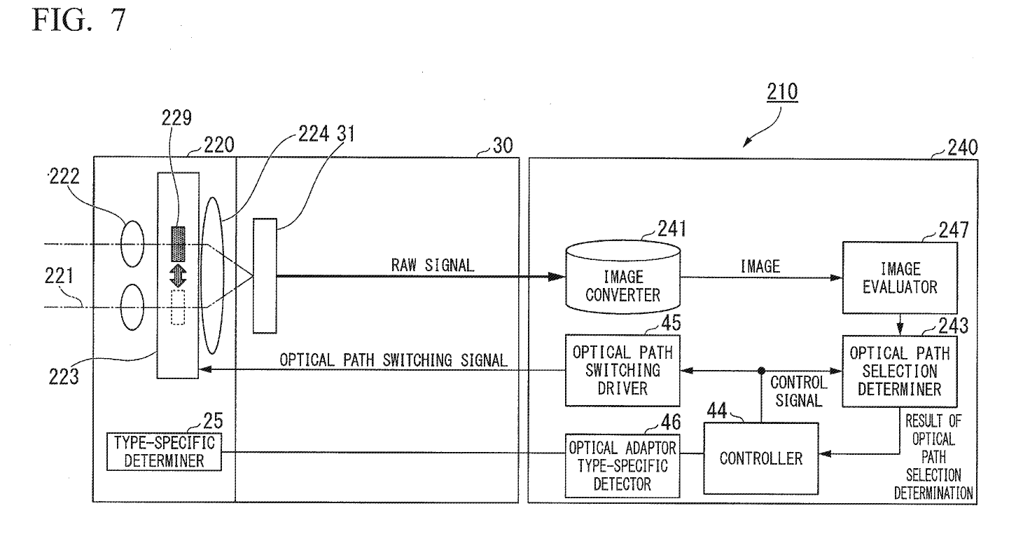

Next, an endoscopic device according to a third embodiment of the present invention will be described. FIG. 7 is a block diagram illustrating a whole constitution of an endoscopic device according to a third embodiment of the present invention. An endoscopic device 210 includes a distal part (an optical adaptor) 220 that is inserted into a specimen, an imaging part 30 that forms an image, and a main body part 240 that controls the entire endoscopic device. The distal part 220 is connected to a distal end of the imaging part 30, and may be attachable/detachable. The imaging part 30 and the main body part 240 are connected by an elongate insertion part (not shown) or the like that is inserted into the specimen. The imaging part 30 includes an imager (an image pickup device) 31.

The distal part 220 includes a first objective optical system 221, a second objective optical system 222, an optical path switching part 223, and a common optical system 224. The distal part 220 may or may not include an optical adaptor type determiner 25. In the distal part 220, the first objective optical system 221 and the second objective optical system 222 have different F-values.

A direction of an optical axis of the first objective optical system 221 and a direction of an optical axis of the second objective optical system 222 are parallel to that of the common optical system 224.

The optical path switching part 223 performs switching between the first objective optical system (the first optical path) 221 and the second objective optical system (the second optical path) 222. That is, the optical path switching part 223 performs switching between a beam of light entering from the first objective optical system 221 and a beam of light entering from the second objective optical system 222, and causes only one of the two beams of light to be incident upon the imager 31 via the common optical system 224. That is, either a first subject image of a subject which is formed by the light entering from the first objective optical system 221 or a second subject image of the subject which is formed by the light entering from the second objective optical system 222 is formed by the imager 31.

The optical path switching part 223 includes, for example, a shading member 229 and cuts off the light entering from the objective optical system on an unselected side by inserting the shading member 229 into the objective optical system on the unselected side. The shading member 229 is driven, for example, by a magnetic drive device.

The image formed by the imager 31 is converted into a RAW signal, and is transmitted to the main body part 240. The main body part 240 includes an image converter 241, an image evaluator 247, an optical path selection determiner 243, a controller 44, and an optical path switching driver 45. The main body part 240 may include an optical adaptor type detector 46.

The image converter 241 converts the RAW signal transmitted from the imager 31, and calculates an image evaluation value of an image. The image evaluator 247 compares image evaluation values of a plurality of images calculated by the image converter 241. To be specific, when an image of a single frame which is formed before the optical path is switched is set as a first image, and when images of two frames which are formed after the optical path is switched are set as second and third images, the image evaluator 247 compares an image evaluation value of the first image and an image evaluation value of the second image, the image evaluation value of the second image and an image evaluation value of the third image, and the image evaluation value of the first image and the image evaluation value of the third image.

The optical path selection determiner 243 determines, on the basis of a result of the comparison between the image evaluation values at the image evaluator 247, to which optical path the optical path is switched now or whether or not the optical path is correctly switched (optical path selection determination), and transmits a result of the optical path selection determination to the controller 44. The controller 44 transmits a control signal for controlling the optical path selection determiner 243 and the optical path switching driver 45 on the basis of the result of the optical path selection determination at the optical path selection determiner 243, and controls the switching of the optical path. The optical path switching driver 45 transmits an optical path switching signal for instructing the optical path switching part 223 to switch the optical path to the optical path switching part 223 on the basis of the control signal from the controller 44.

The optical adaptor type detector 46 reads information (data) about the optical adaptor type determiner 25 of the distal part (the optical adaptor) 220, and determines a type of the distal part (the optical adaptor) 220 on the basis of the data.

Like the second embodiment, in the present embodiment, the image evaluator 247 photographs three images before and after the optical path is switched. However, in the present embodiment, without comparing the amounts of difference between the image data, the image converter 241 calculates the image evaluation value for each image, and the image evaluator 247 compares the amounts of difference between the image evaluation values between the images. The image evaluation value is, for example, an average exposure intensity of an image. Here, the exposure intensity is a product of an incident light intensity and an exposure time. Hereinafter, the image evaluation value will be described as being the average exposure intensity of the image.

The image converter 241 calculates an average exposure intensity of the image of the 0^{th} frame, the image of the 1^{st} frame, and the image of the 2^{nd} frame from an exposure time and gain of the imager 31 at the time of photographing and an average brightness value of the images for each image. Since the first and second objective optical systems 221 and 222 have different F-values, in a case where the optical path switching part 223 operates normally when the optical system is switched from the first objective optical system 221 to the second objective optical system 222 at the boundary between the 0^{th} frame and the 1^{st} frame, an amount of difference between the average exposure intensity of the image of the 1^{st} frame and the average exposure intensity of the image of the 2^{nd} frame is minimized.

On the other hand, in a case the optical path switching part 223 does not operate normally or the optical path has already been switched before being switched, the three images are also obtained by the same optical system. Thus, even if any two of the three images are taken, an amount of difference between any two images is the same as that between other two images.

In this way, when the amounts of difference between the average exposure intensities of the two images selected from the three images are compared, if the amount of difference between the average exposure intensity of the image of the 1^{st} frame and the average exposure intensity of the image of the 2^{nd} frame is minimized, then the optical path selection determiner 243 can determine that the optical system is correctly switched.

When the optical path is switched, the operation of the optical path switching part 223 and the operation of the optical path switching driver 45 are the same as in the second embodiment, and thus description thereof will be omitted.

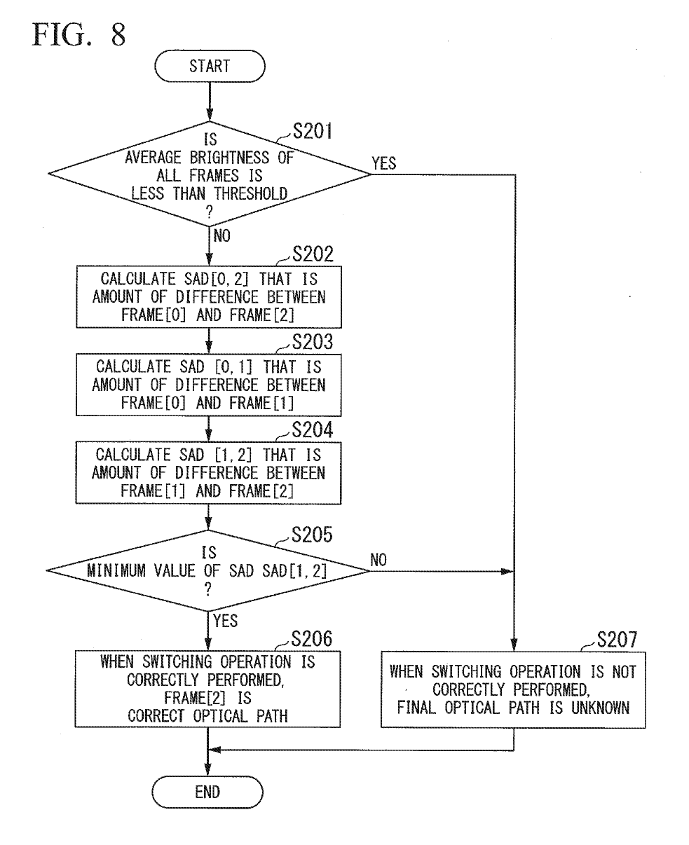

FIG. 8 is a flow chart illustrating an operation of the endoscopic device according to the present embodiment when the optical path is switched. When the optical path switching part 223 switches the optical path at the time of the boundary between the 0^{th} frame and the 1^{st} frame, an operation flow when the optical path is switched is initiated.

First, in step S201, the image evaluator 247 determines whether or not average brightness of the obtained images of all the frames (here, the 0^{th} frame, the 1^{st} frame, and the 2^{nd} frame) is smaller than a prescribed threshold value. A case where the images are completely dark is excluded in step S201.

In a case where the average brightness of the images of all the frames is greater than or equal to the prescribed threshold value, the process proceeds to step S202. In the case where the average brightness of the images of all the frames is smaller than the prescribed threshold value (the images are completely dark), the process proceeds to step S207.

In step S202, the image evaluator 247 calculates ΔY[0,2] that is an amount of difference between the average exposure intensity (the average brightness) of the image of the 0^{th} frame and the average exposure intensity (the average brightness) of the image of the 2^{nd} frame. “Y” means that an average value of brightnesses of points on a y-axial line in an XYZ space is taken. The process proceeds to step S203, and the image evaluator 247 calculates ΔY[0,1] that is an amount of difference between the average exposure intensity (the average brightness) of the image of the 0^{th} frame and the average exposure intensity (the average brightness) of the image of the 1^{st} frame. The process proceeds to step S204, and the image evaluator 247 calculates ΔY[1,2] that is an amount of difference between the average exposure intensity (the average brightness) of the image of the 1^{st} frame and the average exposure intensity (the average brightness) of the image of the 2^{nd} frame.

Next, in step S205, the image evaluator 247 determines whether or not a minimum among the calculated ΔY[0,2], ΔY[0,1], and ΔY[1,2] is ΔY[1,2]. In a case where ΔY[1,2] is the minimum, the process proceeds to step S206, and the optical path selection determiner 243 determines that the switching operation operates normally. Therefore, the image of the 2^{nd} frame is understood be an image that passes through the correct optical path. The operation flow when the optical path is switched is thus completed.

In a case where ΔY[1,2] is not the minimum, the process proceeds to step S207. In step S207, the optical path selection determiner 243 determines that the switching operation is not performed normally. In this case, the current optical path is unknown. The operation flow when the optical path is switched is thus completed.

In the aforementioned description, although the image of the 2^{nd} frame is used in addition to the image of the 0^{th} frame and the image of the 1^{st} frame, the present embodiment is not limited thereto. In addition to enabling the calculation of the amounts of difference between the average exposure intensities of the two images before and after the optical path is switched, it is desirable that the amount of difference between the average exposure intensities of the two images before the optical path is switched or the amount of difference between the average exposure intensities of the two images after the optical path is switched can be calculated.

As described above, in the present embodiment, in the endoscopic device having the optical path switching part, the amounts of difference between the average exposure intensities of the two images selected from among the three images (the first, second, and third images) before and after the optical path is switched are calculated and compared, and it is determined from this result whether or not the optical path is correctly switched. Therefore, even if an additional optical path detecting part is physically mounted, it can be determined whether or not the optical path is correctly switched by image processing. Further, in the present embodiment, since the amounts of difference need not be compared with the prescribed threshold value, the prescribed threshold value need not be adjusted to each subject.

In the aforementioned description, although the amounts of difference between the average exposure intensities of the images which serve as the image evaluation values are compared in the case where the F-values of the first and second objective optical systems are different from each other, the present embodiment is not limited thereto. In a case where focus positions of the first and second objective optical systems are different from each other, the image converter 241 outputs a contrast between images as an image evaluation value. In a case where spectral transmissivities of the first and second objective optical systems are different from each other, the image converter 241 outputs an average hue of images as an image evaluation value. In a case where polarization transmissivities of the first and second objective optical systems are different from each other, the image converter 241 outputs an average brightness of images as an image evaluation value. In a case where brightness shading characteristics of the images of the first and second objective optical systems are different from each other, the image converter 241 outputs a brightness difference of image surroundings as an image evaluation value. In a case where color shading characteristics of the images of the first and second objective optical systems are different from each other, the image converter 241 calculates an average hue of image surroundings as an image evaluation value.

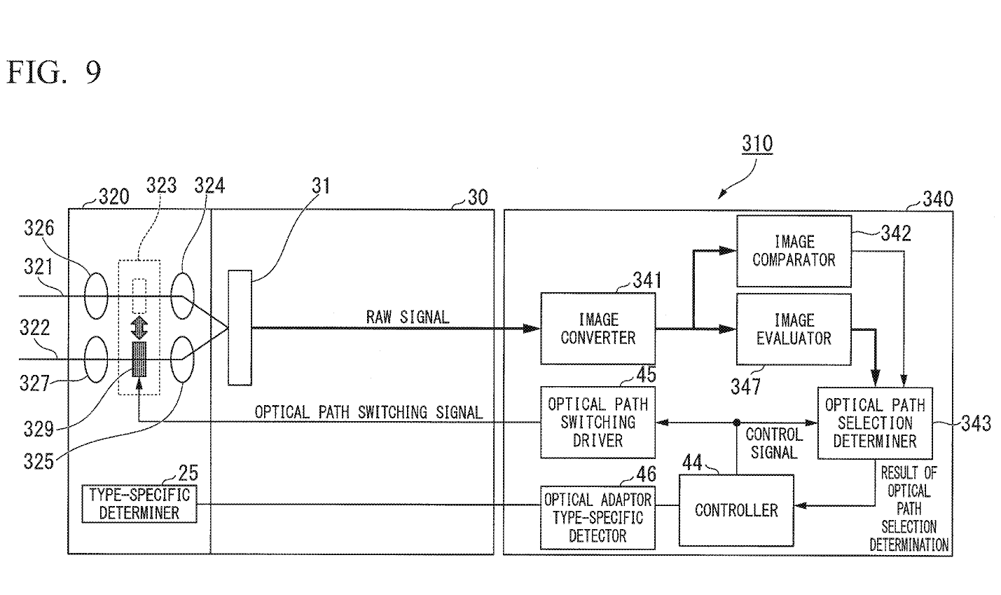

Next, an endoscopic device according to a fourth embodiment of the present invention will be described. FIG. 9 is a block diagram illustrating a whole constitution of an endoscopic device according to a fourth embodiment of the present invention. An endoscopic device 310 includes a distal part (an optical adaptor) 320 that is inserted into a specimen, an imaging part 30 that forms an image, and a main body part 340 that controls the entire endoscopic device. The distal part 320 is connected to a distal end of the imaging part 30, and may be attachable/detachable. The imaging part 30 and the main body part 340 are connected by an elongate insertion part (not shown) or the like that is inserted into the specimen. The imaging part 30 includes an imager (an image pickup device) 31.

The distal part 320 includes a first optical system 321, a second optical system 322, and an optical path switching part 323. The distal part 320 may or may not include an optical adaptor type determiner 25. In the example of FIG. 9, the distal part 320 is a stereo adaptor, and a direction of an optical axis of the first optical system 321 is parallel to that of the second optical system 322. Further, the first optical system 321 includes an objective lens 326 and an eyepiece 324. The second optical system 322 includes an objective lenses 327 and an eyepiece 325.

The optical path switching part 323 performs switching between the first optical system (the first optical path) 321 and the second optical system (the second optical path) 322. That is, the optical path switching part 323 performs switching between a beam of light entering from the first optical system 321 and a beam of light entering from the second optical system 322, and causes only one of the two beams of light to be incident upon the imager 31. That is, either a first subject image of a subject which is formed by the light entering from the first optical system 321 or a second subject image of the subject which is formed by the light entering from the second optical system 322 is formed by the imager 31.

The optical path switching part 323 includes, for example, a shading member 329, and cuts off the light entering from the optical system on an unselected side by inserting the shading member 329 into the optical system on the unselected side. The shading member 329 is driven, for example, by a magnetic drive device.

The image formed by the imager 31 is converted into a RAW signal, and is transmitted to the main body part 340. The main body part 340 includes an image converter 341, an image comparator 342, an image evaluator 347, an optical path selection determiner 343, a controller 44, and an optical path switching driver 45. The main body part 340 may include an optical adaptor type detector 46.

The image converter 341 converts the RAW signal transmitted from the imager 31, and outputs image data to the image comparator 342. Further, the image converter 341 calculates an image evaluation value of an image and outputs the calculated image evaluation value to the image evaluator 347.

The image comparator 342 compares a plurality of images transmitted from the image converter 341. To be specific, when an image of a single frame which is formed before the optical path is switched is set as a first image, and when images of two frames which are formed after the optical path is switched are set as second and third images, the image comparator 342 compares the first image and the second image, the second image and the third image, and the first image and the third image.

The image evaluator 347 compares image evaluation values of a plurality of images transmitted from the image converter 341. To be specific, when an image of a single frame which is formed before the optical path is switched is set as a first image, and when images of two frames which are formed after the optical path is switched are set as second and third images, the image evaluator 347 compares an image evaluation value of the first image and an image evaluation value of the second image, the image evaluation value of the second image and an image evaluation value of the third image, and the image evaluation value of the first image and the image evaluation value of the third image.

The optical path selection determiner 343 determines, on the basis of a result of the comparison between the images at the image comparator 342 and a result of the comparison between the image evaluation values at the image evaluator 347, to which optical path the optical path is switched now or whether or not the optical path is correctly switched (optical path selection determination) and transmits a result of the optical path selection determination to the controller 44. The controller 44 transmits a control signal for controlling the optical path selection determiner 343 and the optical path switching driver 45 on the basis of the result of the optical path selection determination at the optical path selection determiner 343 and controls the switching of the optical path. The optical path switching driver 45 transmits an optical path switching signal for instructing the optical path switching part 323 to switch the optical path to the optical path switching part 323 on the basis of the control signal from the controller 44.

The optical adaptor type detector 46 reads information (data) about the optical adaptor type determiner 25 of the distal part (the optical adaptor) 320 and determines a type of the distal part (the optical adaptor) 320 on the basis of the data.

As in the present embodiment, in a case where the distal part 320 is a stereo adaptor, the first optical system (the right optical system) and the second optical system (the left optical system) have only a slight parallax difference. For this reason, even if the images obtained by the two optical systems (the right optical system and the left optical system) are simply compared, there is almost no difference between the images.

However, even in the stereo adaptor, the two optical systems are different in an angle of incidence upon the imager. Since a main beam angle at each pixel of the imager in the first optical system is different from that in the second optical system, color shading characteristics are different from each other. To be specific, the imager has a sensitivity characteristic corresponding to an angle of incidence of light, and sensitivity decreases when light enters obliquely. Accordingly, a difference in brightness distribution is caused by an inclination of the light entering the imager. That is, in the right optical system (the first optical system) 321, light from the right side is incident upon the imager 31. In this case, an image in which the left side of a field of view is dark is obtained. On the other hand, in the left optical system (the second optical system) 322, light from the left side is incident upon the imager 31. In this case, an image in which the right side of a field of view is dark is obtained. That is, light and dark spots are present in the image, and positions thereof are changed according to the optical system.

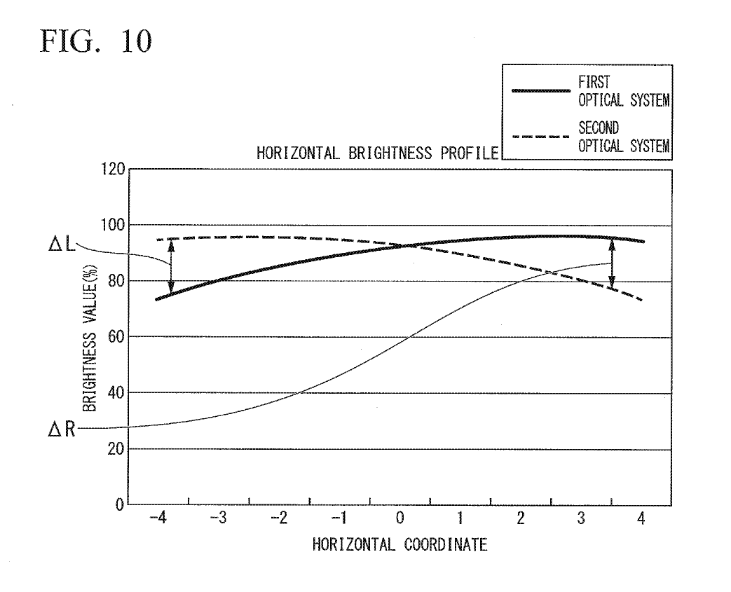

FIG. 10 is a graph illustrating a brightness profile (brightness distribution) of an image in accordance with the present embodiment. The horizontal axis of the graph is a horizontal coordinate, and the vertical axis of the graph is a brightness value. In the graph, an average brightness obtained by averaging brightnesses of points on a vertical line of an image is plotted as a brightness value in each horizontal coordinate. A solid line is a brightness profile of an image which is obtained by the right optical system (the first optical system) 321. A dotted line is a brightness profile of an image which is obtained by the left optical system (the second optical system) 322.

As illustrated in FIG. 10, since the image in which the left side of the field of view is dark is obtained in the right optical system (the first optical system) 321, a brightness profile rises toward the right like the solid line in the graph. On the other hand, since the image in which the right side of the field of view is dark is obtained in the left optical system (the second optical system) 322, a brightness profile falls toward the right side like the dotted line in the graph. In a case where the optical system is normally switched, the brightness profile is switched from the solid line to the dotted line in the graph.

Therefore, a result of calculating the value obtained by averaging the brightnesses of the points on the vertical line at a left end position of the horizontal coordinate is set as an image left end brightness. A result of calculating the value obtained by averaging the brightnesses of the points on the vertical line at a right end position of the horizontal coordinate is set as an image right end brightness. The image left end brightness and the image right end brightness are used as image evaluation values.

Like the second and third embodiments, in the present embodiment, the image evaluator 347 photographs three images before and after the optical path is switched. However, in the present embodiment, in addition to the amounts of difference between the image data, the image evaluator 347 compares the amounts of difference between the image evaluation values between the images. Further, in the present embodiment, the image evaluation values are the image left end brightness and the image right end brightness of each image.

That is, the image evaluator 347 compares the image evaluation value of the first image and the image left end brightness and the image right end brightness of the second image, the image evaluation value of the second image and the image left end brightness and the image right end brightness of the third image, and the image evaluation value of the first image and the image left end brightness and the image right end brightness of the third image.

Regardless of how far a subject is apart, a difference between the image obtained by the aforementioned right optical system and the image obtained by the aforementioned left optical system occurs. Further, this difference occurs only in a case where the two optical systems are connected side by side to the same imager. That is, the present embodiment can be applied only to two optical systems having parallaxes. The present embodiment cannot be applied to two optical systems based on insertion/ejection of an optical member in the course of an optical path. Further, when a subject is completely dark, the subject cannot be detected, and the present embodiment cannot be applied.

A case where the optical system is switched from the first optical system 321 to the second optical system 322 at the time of the boundary between the 0^{th} frame and the 1^{st} frame will be described. When an amount of difference between image data of an m-th frame and image data of an n-th frame according to SAD is expressed as SAD[m,n], the image comparator 342 calculates SAD[0,1], SAD[0,2], and SAD[1,2]. The image comparator 342 determines whether or not a minimum among the calculated SAD[0,1], SAD[0,2], and SAD[1,2] is SAD[1,2]. In a case where the SAD[1,2] is the minimum, the optical path selection determiner 343 determines that the switching operation is performed normally.

On the other hand, in a case where the optical path switching part 323 does not operate normally or the optical path has already been switched before being switched, the three images are obtained by the same optical system, and thus SAD[0,1], SAD[0,2], and SAD[1,2] have the same values.

When an amount of difference between an image left end brightness of an image of the m-th frame and an image left end brightness of an image of the n-th frame is expressed as ΔL[m,n], and when an amount of difference between an image right end brightness of the image of the m-th frame and an image right end brightness of the image of the n-th frame is expressed as ΔR[m,n], the image evaluator 347 calculates ΔL[0,1], ΔL[0,2], and ΔL[1,2]. The image comparator 342 determines whether or not a minimum among the calculated ΔL[0,1], ΔL[0,2], and ΔL[1,2] is ΔL[1,2]. In a case where ΔL[1,2] is the minimum, the optical path selection determiner 343 determines that the switching operation is performed normally.

On the other hand, in a case where the optical path switching part 323 does not operate normally or the optical path has already been switched before being switched, the three images are obtained by the same optical system, and ΔL[0,1], ΔL[0,2], and ΔL[1,2] have the same values.

When the amount of difference between the image right end brightness of the image of the m-th frame and the image right end brightness of the image of the n-th frame is expressed as ΔR[m,n], the image evaluator 347 calculates ΔR[0,1], ΔR[0,2], and ΔR[1,2]. The image comparator 342 determines whether or not a minimum among the calculated ΔR[0,1], ΔR[0,2], and ΔR[1,2] is ΔR[1,2]. In a case where ΔR[1,2] is the minimum, the optical path selection determiner 343 determines that the switching operation is performed normally.

On the other hand, in a case where the optical path switching part 323 does not operate normally or the optical path has already been switched before being switched, the three images are obtained by the same optical system, and ΔR[0,1], ΔR[0,2], and ΔR[1,2] have the same values.

When the optical path is switched, the operation of the optical path switching part 323 and the operation of the optical path switching driver 45 are the same as in the second and third embodiments, and thus description thereof will be omitted.

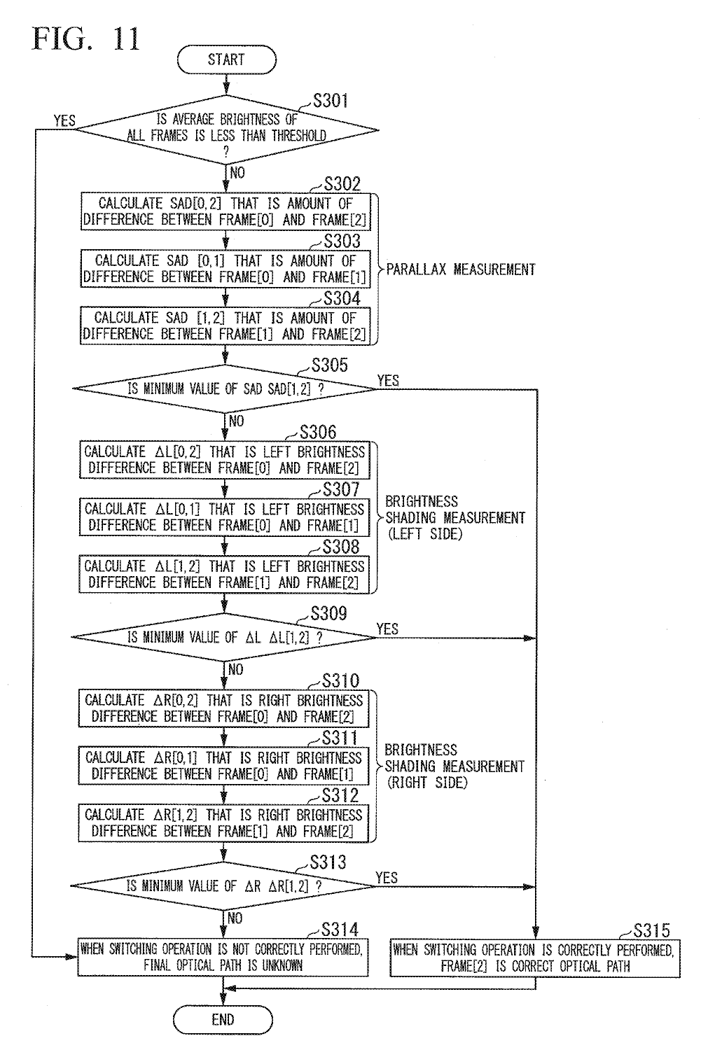

FIG. 11 is a flow chart illustrating an operation of the endoscopic device according to the present embodiment when the optical path is switched. When the optical path switching part 323 switches the optical path at the time of the boundary between the 0^{th} frame and the 1^{st} frame, an operation flow when the optical path is switched is initiated.

First, in step S301, the image comparator 342 determines whether or not average brightness of the obtained images of all the frames (here, the 0^{th} frame, the 1^{st} frame, and the 2^{nd} frame) is smaller than a prescribed threshold value. A case where the images are completely dark is excluded in step S301.

In a case where the average brightness of the images of all the frames is greater than or equal to the prescribed threshold value, the process proceeds to step S302. In the case where the average brightness of the images of all the frames is smaller than the prescribed threshold value (the images are completely dark), the process proceeds to step S314.

In step S302, the image comparator 342 calculates SAD[0,2] that is an amount of difference between image data of the 0^{th} frame and image data of the 2^{nd} frame according to SAD. The process proceeds to step S303, and the image comparator 342 calculates SAD[0,1] that is an amount of difference between image data of the 0^{th} frame and image data of the 1^{st} frame according to SAD. This process proceeds to step S304, and the image comparator 342 calculates SAD[1,2] that is an amount of difference between the image data of the 1^{st} frame and image data of the 2^{nd} frame according to SAD.

Next, in step S305, the image comparator 342 determines whether or not a minimum among the calculated SAD[0,2], SAD[0,1], and SAD[1,2] is SAD[1,2]. In a case where SAD[1,2] is the minimum, the process proceeds to step S315. In a case where SAD[1,2] is not the minimum, the process proceeds to step S306.

In step S306, the image evaluator 347 calculates ΔL[0,2] that is an amount of difference between the image left end brightness of the image of the 0^{th} frame and the image left end brightness of the image of the 2^{nd} frame. The process proceeds to step S307, and the image evaluator 347 calculates ΔL[0,1] that is an amount of difference between the image left end brightness of the image of the 0^{th} frame and the image left end brightness of the image of the 1^{st} frame. The process proceeds to step S308, and the image evaluator 347 calculates ΔL[1,2] that is an amount of difference between the image left end brightness of the image of the 1^{st} frame and the image left end brightness of the image of the 2^{nd} frame.

Next, in step S309, the image evaluator 347 determines whether or not a minimum among the calculated ΔL[0,2], ΔL[0,1], and ΔL[1,2] is ΔL[1,2]. In a case where ΔL[1,2] is the minimum, the process proceeds to step S315. In a case where ΔL[1,2] is not the minimum, the process proceeds to step S310.

In step S310, the image evaluator 347 calculates ΔR[0,2] that is an amount of difference between the image right end brightness of the image of the 0^{th} frame and the image right end brightness of the image of the 2^{nd} frame. The process proceeds to step S311, and the image evaluator 347 calculates ΔR[0,1] that is an amount of difference between the image right end brightness of the image of the 0^{th} frame and the image right end brightness of the image of the 1^{st} frame. The process proceeds to step S312, and the image evaluator 347 calculates ΔR[1,2] that is an amount of difference between the image right end brightness of the image of the 1^{st} frame and the image right end brightness of the image of the 2^{nd} frame.

Next, in step S313, the image evaluator 347 determines whether or not a minimum among the calculated ΔR[0,2], ΔR[0,1], and ΔR[1,2] is ΔR[1,2]. In a case where ΔR[1,2] is the minimum, the process proceeds to step S315. In a case where ΔR[1,2] is not the minimum, the process proceeds to step S314.

In step S314, the optical path selection determiner 343 determines that the switching operation is not performed normally. In this case, the current optical path is unknown. The operation flow when the optical path is switched is thus completed.

In step S315, the optical path selection determiner 343 determines that the switching operation is performed normally. Therefore, the image of the 2^{nd} frame is understood be an image that passes through the correct optical path. The operation flow when the optical path is switched is thus completed.

In the aforementioned operation flow, the evaluation based on the parallax measurement (S302 to S305), the evaluation based on the left brightness shading measurement (S306 to S309), and the evaluation based on the right brightness shading measurement (S310 to S313) are performed in this order, the flow is completed at a point in time when it can be determined that there is a difference between the images of the two optical systems, and it is determined that the switching operation of the optical path is performed normally. However, the present embodiment is not limited thereto. The order in which these three evaluations are performed is arbitrarily combined. The operation flow may include one or two of the three evaluations. Further, in a case where it can be determined that there is a difference between the images of the two optical systems in two or more of the three evaluations, it may be determined that the switching operation of the optical path is performed normally.

In the above description, the image of the 2^{nd} frame is used in addition to the image of the 0^{th} frame and the image of the 1^{st} frame, the present embodiment is not limited thereto. In addition to enabling the calculation of the amounts of difference between the two image data before and after the optical path is switched, the image left end brightness, and the image right end brightness, it is desirable that the amount of difference between the two image data before the optical path is switched, the image left end brightness, and the image right end brightness or the amount of difference between the two image data, the image left end brightness, and the image right end brightness after the optical path is switched can be calculated.

As described above, in the present embodiment, the endoscopic device having the optical path switching part, the amounts of difference between the image data of the two images selected from among the three images (the first, second, and third images) before and after the optical path is switched, the image left end brightness, and the image right end brightness are calculated, and the amounts of difference are compared. Then, tt is determined from this result whether or not the optical path is correctly switched. Therefore, even if no additional optical path detecting part is physically mounted, it can be determined whether or not the optical path is correctly switched by image processing. Further, in the present embodiment, since the amounts of difference need not be compared with the prescribed threshold value, the prescribed threshold value need not be adjusted to each subject.

In the aforementioned embodiment, the image evaluation value based on the brightness profile of the image is used. Brightness of an image varies according to a wavelength of light. When the wavelength of light varies, distribution of colors also varies. Therefore, even if a color-difference profile is used instead of the brightness profile, the image obtained by the first optical system and the image obtained by the second optical system are different in profile slope (color shading characteristic). To be specific, since the directions of beams of light entering from right and left sides of an image formation area are different, a white balance deviates on right and left sides of the image, but a peripheral portion of the image has a greater color difference than a central portion of the image. Here, an average color of right and left ends of the image obtained by the first optical system is compared with that of the image obtained by the second optical system, and thereby it can be determined whether or not the optical path is correctly switched. In this way, the present embodiment may use the image evaluation value based on the color-difference profile of the average color.

Each embodiment of the present invention can also be applied to the case where the optical adaptor type determiner is present or otherwise. That is, the main body part of the endoscopic device may or may not know a type of the distal part (the optical adaptor). In a case where the optical adaptor type determiner is present, it is known what kind of difference occurs at the two optical systems, and thus an evaluating method corresponding to this may be adopted. In a case where it is not known which is attached, various evaluating methods may be adopted.

In each embodiment, the endoscopic device having a constitution in which the distal part (the optical adaptor) including the two optical systems can be separated from a distal end side of the imaging part has been described. However, each embodiment of the present invention is not limited to this constitution, and may be a constitution in which the distal part and the imaging part are integrally formed.

Each component that is shown in each embodiment and is included in the main body part is for describing a function and processing related thereto. In the endoscopic device, the functions and processing related to the plurality of components described in each embodiment may be simultaneously realized by one component. For example, functions and processing of the image comparator 42 and the optical path selection determiner 43 illustrated in FIG. 1 may be performed by the controller 44.

Further, the components that are shown in each embodiment and are included in the main body part, for example, the image converter 41, the image comparator 42, the optical path selection determiner 43, the controller 44, the optical path switching driver 45, and the optical adaptor type detector 46 illustrated in FIG. 1 may be realized independently or as a whole by a computer that is constituted of one or a plurality of processors, a logic circuit, a memory, an input/output interface, a computer-readable recording medium, and so on. In this case, the components may be realized by recording a program for realizing the function of each component or the entire main body part on the recording medium and reading and executing the recorded program into a computer system. For example, the processor is at least one of a CPU, a digital signal processor (DSP), and a graphics processing unit (GPU). For example, the logic circuit is at least one of an application specific integrated circuit (ASIC) and a field-programmable gate array (FPGA).

For example, the aforementioned various functions and processing related to the endoscopic device of the present embodiment may be performed by recording the program for realizing the function and processing of the endoscopic device, such as the functions and processing of the main body part 40 illustrated in FIG. 1 or parts thereof, the image converter 41, the image comparator 42, the optical path selection determiner 43, the controller 44, the optical path switching driver 45, the optical adaptor type detector 46 included in the main body part 40, and so on, on the computer-readable recording medium, and reading and executing the program recorded on the recording medium in the computer system. The “computer system” used herein may include OS or hardware such as peripheral devices. Further, the “computer system” includes a home page providing environment (or a display environment) if it uses a WWW system. Further, the “computer-readable recording medium” refers to a storage device such as a flexible disk, a magneto-optical disk, a ROM, a writable nonvolatile memory such as a flash memory, a portable medium such as a CD-ROM, a hard disk embedded in the computer system, and so on.

Furthermore, the “computer-readable recording medium” includes a storage device for holding a program for a fixed time such as a volatile memory inside a computer system (e.g., a dynamic random access memory (DRAM)) that serves as a server or a client when a program is sent through a network such as the Internet or a communication line such as a telephone line. Further, the program may be transmitted to another computer system from the computer system that stores the program in a storage device or the like through a transmission medium or a transmission wave out of the transmission medium. Here, the “transmission medium” that transmits the program refers to a medium having a function of transmitting information, such as a network (a communication network) such as the Internet or a communication line (a communication wire) such as a telephone line. Further, the program may implement some of the aforementioned functions. Furthermore, the program may be a so-called differential file (a differential program) that is realized by combining the aforementioned functions with the program that is previously stored in the computer system.

The words indicating directions such as “forward, backward, above, below, right, left, vertical, horizontal, longitudinal, transverse, row, and column” in this specification are used to describe these directions in the device of the present invention. Therefore, these words used to describe the specification of the present invention should be relatively interpreted in the device of the present invention.

While preferred embodiments of the present invention have been described, the present invention is not limited to these embodiments and their modifications. Additions, omissions, substitutions, and other modifications of the constitution can be made without departing from the scope of the present invention.

What is claimed is:

1. An endoscopic device comprising:
a switching part configured to switch optical systems such that only any one of a first subject image of a subject which is formed by light passing through a first optical system and a second subject image of the subject which is formed by light passing through a second optical system is formed in an image formation area in which the first subject image and the second subject image are formed in common;
an image pickup device configured to photograph the first and second subject images formed in the image formation area to generate an image;
a driver configured to output a switching signal to the switching part, the switching signal being configured to switch the first optical system to the second optical system or switch the second optical system to the first optical system; and
a controller,
wherein the controller is configured to
give the driver an instruction to output the switching signal,
after the instruction is given, compare a plurality of images generated by the image pickup device before and after the instruction is given, and
determine on the basis of a result of the comparison whether or not the switching is performed normally by the switching part.

2. The endoscopic device according to claim 1, wherein the controller is configured to
calculate, after the instruction is given, an amount of difference between a first image generated by the image pickup device before the instruction is given and a second image generated by the image pickup device after the instruction is given,
compare the amount of difference and a prescribed threshold value, and
in a case where the amount of difference is greater than the prescribed threshold value, determine that the switching is performed normally by the switching part.

3. The endoscopic device according to claim 1, wherein, after the instruction is given, the controller compares a first image generated by the image pickup device before the instruction is given, a second image generated by the image pickup device after the instruction is given, and a third image generated by the image pickup device after the second image is generated.

4. The endoscopic device according to claim 3, wherein the controller is configured to
compare an amount of difference between the first image and the second image, an amount of difference between the first image and the third image, and an amount of difference between the second image and the third image, and
in a case where the amount of difference between the second image and the third image is a minimum, determine that the switching is performed normally by the switching part.

5. The endoscopic device according to claim 1, wherein, after the instruction is given, the controller compares a first image generated by the image pickup device before the instruction is given, a second image generated by the image pickup device after the instruction is given, and a third image generated by the image pickup device before the first image is generated.

6. The endoscopic device according to claim 5, wherein the controller is configured to
compare an amount of difference between the first image and the second image, an amount of difference between the first image and the third image, and an amount of difference between the second image and the third image, and
in a case where the amount of difference between the first image and the second image is a minimum, determine that the switching is performed normally by the switching part.

7. The endoscopic device according to claim 1, wherein, after the instruction is given, the controller compares an average brightness value of the images generated by the image pickup device before and after the instruction is given with a prescribed threshold value, and in a case where the average brightness value of all the images is smaller than the prescribed threshold value, determines that the switching is not performed normally by the switching part.

8. The endoscopic device according to claim 1, wherein, after the instruction is given, the controller compares contrasts of the images generated by the image pickup device before and after the instruction is given.

9. The endoscopic device according to claim 1, wherein, after the instruction is given, the controller compares average exposure intensities of the images generated by the image pickup device before and after the instruction is given.

10. The endoscopic device according to claim 1, wherein, after the instruction is given, the controller compares average hues of the images generated by the image pickup device before and after the instruction is given.

11. The endoscopic device according to claim 1, wherein, after the instruction is given, the controller compares left or right average brightnesses of the images generated by the image pickup device before and after the instruction is given.

12. The endoscopic device according to claim 1, wherein, after the instruction is given, the controller compares left or right average colors of the images generated by the image pickup device before and after the instruction is given.

13. The endoscopic device according to claim 1, wherein the controller is configured to
compare, after the instruction is given, two or more of average exposure intensities, contrasts, average hues, left or right average brightness, and left or right average colors of the images generated by the image pickup device before and after the instruction is given, and
determine, on the basis of a result of the comparison, whether or not the switching is performed normally by the switching part.

14. The endoscopic device according to claim 1, wherein:
a distal part of the endoscopic device having the first and the second optical systems includes a type determiner that includes information for identifying a type; and
the endoscopic device further includes a type detector that reads information about the type determiner and determines a type of the distal part.

15. The endoscopic device according to claim 14, wherein, in a case where the type detector determines that the first subject image and the second subject image are different in focal position from each other in the distal part,
after the instruction is given, the controller compares contrasts of the images generated by the image pickup device before and after the instruction is given.

16. The endoscopic device according to claim 14, wherein, in a case where the type detector determines that the first subject image and the second subject image are different in aperture value from each other in the distal part,
after the instruction is given, the controller compares average exposure intensities of the images generated by the image pickup device before and after the instruction is given.

17. The endoscopic device according to claim 14, wherein, in a case where the type detector determines that the first subject image and the second subject image have respective parallaxes in the distal part, light from the first optical system is obliquely incident upon the image formation area from a right side, and light from the second optical system is obliquely incident upon the image formation area from a left side,
after the instruction is given, the controller compares left or right average brightnesses of the images generated by the image pickup device before and after the instruction is given.

18. The endoscopic device according to claim 14, wherein, in a case where the type detector determines that the first optical system and the second optical system are different in spectral transmissivity from each other in the distal part,
after the instruction is given, the controller compares average colors of the images generated by the image pickup device before and after the instruction is given.

19. The endoscopic device according to claim 14, wherein, in a case where the type detector determines that the first optical system and the second optical system are different in polarization transmissivity from each other in the distal part,
after the instruction is given, the controller compares average brightnesses of the images generated by the image pickup device before and after the instruction is given.

20. The endoscopic device according to claim 14, wherein, in a case where the type detector determines that the first subject image and the second subject image have respective parallaxes in the distal part, light from the first optical system is obliquely incident upon the image formation area from a right side, and light from the second optical system is obliquely incident upon the image formation area from a left side,
after the instruction is given, the controller compares left or right average colors of the images generated by the image pickup device before and after the instruction is given.

21. The endoscopic device according to claim 1, wherein the controller further includes a storage that stores the images generated by the image pickup device before and after the instruction is given.

22. A method of determining switching of optical systems in an endoscopic device that includes
a switching part configured to switch the optical systems such that only any one of a first subject image of a subject which is formed by light passing through a first optical system and a second subject image of the subject which is formed by light passing through a second optical system is formed in an image formation area in which the first subject image and the second subject image are formed in common, and
an image pickup device configured to photograph the first and second subject images formed in the image formation area to generate an image,
the method comprising:
a switching step of instructing the switching part to switch the first optical system to the second optical system or to switch the second optical system to the first optical system;
a step of, after the switching step, comparing a plurality of images generated by the image pickup device before and after the switching step; and
a step of determining on the basis of a result of the comparison whether or not the switching is performed normally by the switching part.

23. A computer-readable recording medium that records a program for controlling an endoscopic device that includes
a switching part configured to switch optical systems such that only any one of a first subject image of a subject which is formed by light passing through a first optical system and a second subject image of the subject which is formed by light passing through a second optical system is formed in an image formation area in which the first subject image and the second subject image are formed in common,
an image pickup device configured to photograph the first and second subject images formed in the image formation area to generate an image, and
a controller,
wherein the program causes the controller of the endoscopic device to execute:
a switching step of instructing the switching part to switch the first optical system to the second optical system or to switch the second optical system to the first optical system;
a step of, after the switching step, comparing a plurality of images generated by the image pickup device before and after the switching step; and
a step of determining on the basis of a result of the comparison whether or not the switching is performed normally by the switching part.
